(19) United States
(12) Patent Application Publication
(10) Pub. No.: US 2019/0134003 A1
(43) Pub. Date: May 9, 2019

(54) COMBINATION THERAPIES USING INDAZOLYLBENZAMIDE DERIVATIVES FOR THE TREATMENT OF CANCER

(71) Applicant: ESANEX, INC., Indianapolis, IN (US)

(72) Inventor: Everardus O. M. ORLEMANS, Chapel Hill, NC (US)

(21) Appl. No.: 16/302,084

(22) PCT Filed: May 18, 2017
(86) PCT No.: PCT/US2017/033229

§ 371 Date: Nov. 15, 2018

Related U.S. Application Data
(60) Provisional application No. 62/338,370, filed on May 18, 2016.

(51) Int. Cl. A61K 31/416; A61K 31/282; A61K 31/337; A61P 35/00

(52) U.S. Cl. CPC A61K 31/416; A61P 35/00; A61K 31/337; A61K 31/282

(57) ABSTRACT

The invention relates to combination therapies useful in the treatment and/or prevention of cancer.

### CROSS-REFERENCE TO RELATED APPLICATIONS

This application claims priority to U.S. Provisional Application No. 62/338,370, filed May 18, 2016, the disclosure of which is hereby incorporated by reference in its entirety.

### BACKGROUND OF THE INVENTION

### Field of the Invention

The invention relates to combination therapies useful in the treatment and/or prevention of diseases and/or conditions related to cell proliferation, such as cancer.

### Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989)). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991)). Under conditions of unregulated angiogenesis, therapeutic methods designed to control, repress, and/or inhibit angiogenesis could lead to the abrogation or mitigation of these conditions and diseases.

Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock protein 90 (Hsp90) chaperone proteins stabilize well over 200 different known client proteins helping them to fold correctly as they take up their rightful positions in the cell. Inhibitors of the chaperone protein Hsp90 are of current interest because of the central role of Hsp90 in the maturation and maintenance of numerous proteins that are critical for tumor cell viability and growth. Possible, relevant Hsp90 clients for the tumor types under investigation include mutated STK11/LKB1 (Boudeau, J. et al. Biochem. J. 370, 849-857 (2003)) and NF1 null (De Raedt, T. et al. Cancer Cell 20(3), 400-413 (2011)) in the NSCLC population, and DNA methyltransferase-1 (Yamaki, H., et al. J Antibiot (Tokyo) 64(9), 635-44 (2011)) in the SCLC population.

Lung cancer is the leading cause of cancer death, annually resulting in more than one million deaths worldwide. About 1.2 million new cases are diagnosed each year and prognoses are poor. Lung adenocarcinoma is the most common form of lung cancer and has an average 5-yr survival rate of 15%, mainly because of late-stage detection and a paucity of late-stage treatments. Therapeutic progresses have signed out the last decade, but median survival for patients in advanced stage is still disappointing.

Small cell lung cancer (SCLC) accounts for about 15% of all lung cancers. The prognosis of SCLC patients is devastating and no biologically targeted therapeutics are active in this tumor type. The majority of SCLC tumors possess a RB null phenotype (Wistuba I, et al., Semin. Oncol., 28 (2 Suppl 4), 3-13 (2001)). SCLC patients have been treated with a combination of carboplatin plus paclitaxel with the majority of responses being observed in SCLC patients having received up to 4 cycles of the combination (Thomas, P. et al., J Clin Oncol 19, 1320-1325 (2001)). Remissions with cis- or carboplatin combinations are observed initially, but oftentimes resistance occurs resulting in a more difficult to treat tumor.

For stage III/IV Non-Small Cell Lung Cancer (NSCLC), platinum-based combined chemotherapy is the current standard of care, but with much room for improvement (Azzoli, C. et al., J. Oncol. Pract. 8(1), 63-66 (2012)). In patients with stage IV NSCLC, first-line cytotoxic chemotherapy should be stopped at disease progression or after four cycles in patients whose disease is not responding to treatment (Azzoli C. et al., J Clin Oncol 27, 6251-6266 (2009)).

### SUMMARY OF THE INVENTION

The inventors have discovered that the Hsp90 inhibitors of this disclosure in combination with carboplatin and paclitaxel were efficient in treatment of cancer.

In one aspect, the disclosure provides methods for treating cancer in a subject in need thereof, the method comprising administering to the subject:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, wherein the Hsp90 inhibitor is administered in an amount of about 50 mg/m² to about 150 mg/m²;
b) carboplatin administered in an amount sufficient to result in a target Area under Curve (AUC) of about 2 to about 7; and
c) paclitaxel administered in an amount of about 100 mg/m² to about 225 mg/m².

In another aspect, the disclosure provides methods for treating cancer in a subject in need thereof, the method comprising administering a therapeutically effective amount of:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof;
b) carboplatin; and
c) paclitaxel,
   to the subject on a dosage schedule, wherein the dosage schedule comprises four or six 28-day treatment cycles, and wherein:
(i) the Hsp90 inhibitor is administered on alternating days, i.e., every other day, for at least 21 days during each 28-day treatment cycle; and
(ii) carboplatin and paclitaxel are administered every 21±2 days starting on day 2 or day 4 of the first 28-day treatment cycle.
   In one embodiment of this aspect, the Hsp90 inhibitor is administered in an amount of about 50 mg/m² to about 150 mg/m²; carboplatin is administered in an amount sufficient to result in a target Area under Curve (AUC) of about 2 to about 7; and paclitaxel is administered in an amount of about 100 mg/m² to about 225 mg/m².

### BRIEF DESCRIPTION OF THE DRAWINGS

FIG. 1 is a chart showing a dosing schedule of the disclosure.

### DETAILED DESCRIPTION OF THE INVENTION

Before the disclosed methods are described, it is to be understood that the aspects described herein are not limited to specific embodiments, or compositions, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivative or pro-drug, such as pharmaceutically acceptable salts and esters. The manufacture of pharmaceutical compositions for different routes of administration falls within the capabilities of a person skilled in medicinal chemistry.

In view of the present disclosure, the methods described herein can be configured by the person of ordinary skill in the art to meet the desired need. In general, the disclosed methods provide improvements in the treatment of cancer. Surprisingly, the inventors have found that the methods of the disclosure were more efficient in treatment of cancer. For example, when patients with NSCLC were treated with the methods of the disclosure, of the evaluable patients (N=18; with RECIST (Response Evaluation Criteria In Solid Tumors, typically measured by CT scan or MRI) 39% had partial response rate and 56% had stable disease. In contrast, when the patients with NSCLC were treated with carboplatin and paclitaxel only, without SNX-5422, about 25% patients had partial response rate and 24% patients had stable disease. In addition, of evaluable patients, 67% of patients treated with the methods of the disclosure went onto maintenance, whereas only 57% of patients treated with carboplatin and paclitaxel only went onto maintenance. For evaluable patients (N=17; with Principal Investigator assessment) the median progression-free survival estimate was found to be 7.1 months compared to only 5.8 months for patients with NSCLC treated with carboplatin and paclitaxel only.

The methods of the disclosure are particularly useful in treatment of lung, esophageal, ovarian, head-and-neck, mesothelioma, melanoma, testicular, stomach, bladder, uterine, colon, prostate, renal cell, pancreatic, and neuroendocrine cancer. In some embodiments, the methods of the disclosure are used in treatment of lung cancer. In other embodiments, the methods of the disclosure are used in treatment of non-small cell lung cancer (NSCLC).

In some embodiments of this disclosure, the subject in need is a human subject or patient. In some embodiments the subject, e.g., a human, has been previously treated with an anticancer therapy (e.g., surgery, chemotherapy, radiation therapy, hormonal therapy, and Immunotherapy). In some other embodiments the subject has not been previously treated with an anticancer therapy.

The methods of the disclosure require an Hsp90 inhibitor or a pharmaceutically acceptable salt thereof. In some embodiments, the Hsp90 inhibitor is 4-(6,6-dimethyl-4-oxo-3-trifluoromethyl-4,5,6,7-tetrahydro-indazol-1-yl)-2-(trans-4-hydroxy-cyclohexylamino)-benzamide:

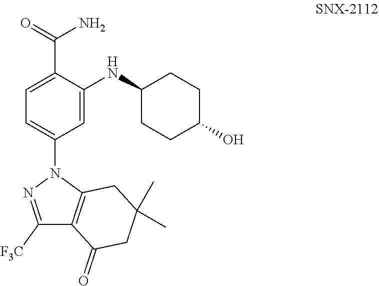 [SMILES: CC1(C)CC(=O)c2c(C(F)(F)F)nn(-c3ccc(C(N)=O)c(N[C@H]4CC[C@H](O)CC4)c3)c2C1]

or a pharmaceutically acceptable salt thereof. Synthesis and characterization data for SNX-2112 is described in U.S. Pat. No. 7,358,370, which is incorporated by reference in its entirety.

In some embodiments, the Hsp90 inhibitor is trans-4-{(2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate:

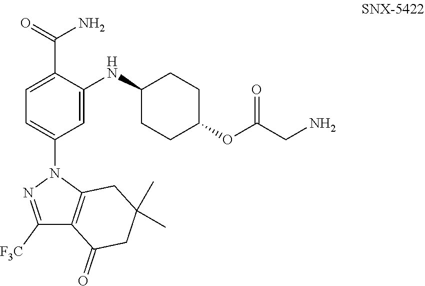 [SMILES: CC1(C)CC(=O)c2c(C(F)(F)F)nn(-c3ccc(C(N)=O)c(N[C@H]4CC[C@H](OC(=O)CN)CC4)c3)c2C1]

or a pharmaceutically acceptable salt thereof. Synthesis and characterization data for SNX-5422 is described in U.S. Pat. No. 7,358,370, which is incorporated by reference in its entirety.

In the methods of the disclosure the Hsp90 inhibitor is administered in an amount of about 50 mg/m² to about 150 mg/m², or about 50 mg/m² to about 100 mg/m², or about 75 mg/m² to about 100 mg/m². In one embodiment, the Hsp90 inhibitor is administered in an amount that gradually increases from about 50 mg/m² to about 100 mg/m². In another embodiment, the Hsp90 inhibitor is administered in an amount of about 100 mg/m². In another embodiment, the Hsp90 inhibitor is administered in an amount of about 75 mg/m². In another embodiment, the Hsp90 inhibitor is administered in an amount of about 50 mg/m².

In the methods of the disclosure, carboplatin is administered in an amount sufficient to result in a target Area under Curve (AUC) of about 2 to about 7, In some embodiments, carboplatin is administered in an amount sufficient to result in a target Area under Curve (AUC) of about 3 to about 6, or a target AUC of about 3 to about 5, or a target AUC of about 4 to about 5, or a target AUC of about 5.

AUC is calculated based on Calvert formula and the actual measurements of Glomerular Filtration Rate (GFR):

Total Carboplatin Dose (mg)=(target AUC)×(GFR+25) Calvert formula:

Maximum Carboplatin Dose (mg)=target AUC (mg·min/mL)×(150 mL/min)

The maximum dose is based on a GFR estimate that is capped at 125 mL/min for patients with normal renal function. E.g.,
for a target AUC=6, the maximum dose is 6×150=900 mg
for a target AUC=5, the maximum dose is 5×150=750 mg
for a target AUC=4, the maximum dose is 4×150=600 mg

In the methods of the disclosure, paclitaxel is administered in an amount of about 150 mg/m² to about 225 mg/m², or about 150 mg/m² to about 200 mg/m², or about 160 mg/m² to about 200 mg/m², or about 160 mg/m² to about 190 mg/m², or about 160 mg/m² to about 180 mg/m², or about 170 mg/m² to about 180 mg/m², or about 172 mg/m² to about 177 mg/m². In some embodiments, paclitaxel is administered in an amount of about 175 mg/m².

In an exemplary, non-limiting embodiment of the methods of the disclosure, the Hsp90 inhibitor is administered in an amount of about 100 mg/m²; carboplatin is administered in an amount sufficient to result in target Area under Curve (AUC) of about 5; and paclitaxel is administered in an amount of about 170 mg/m² to about 180 mg/m².

In some methods of the disclosure, the Hsp90 inhibitor, carboplatin, and paclitaxel may be administered simultaneously, separately, or sequentially in the methods of the disclosure. Also, after the first 28-day cycle, either carboplatin or paclitaxel may be eliminated from the treatment regimen so that only one of paclitaxel or carboplatin is administered at the predetermined time as described herein.

In other methods of the disclosure, the Hsp90 inhibitor, carboplatin, and paclitaxel are administered according to the dosage schedule. In one embodiment, the dosage schedule comprises four or six 28-day treatment cycles, and wherein:
(i) the Hsp90 inhibitor is administered on alternating for at least 21 days during each 28-day treatment cycle; and
(ii) carboplatin and paclitaxel are administered every 21±2 days starting on day 2 or day 4 of the first 28-day treatment cycle.
   In one embodiment, the dosage schedule comprises four 28-day treatment cycles. In another embodiment, the dosage schedule comprises six 28-day treatment cycles.

In certain embodiments, the Hsp90 inhibitor is administered on alternating days over a period of 21, 22, 23, 24, 25, 26, or 27 days, and particularly for 21 days, within each 28-day treatment cycle,

Carboplatin and paclitaxel may be administered starting on day 2 of the first 28-day treatment cycle. In another embodiment, carboplatin and paclitaxel may be administered starting on day 4 of the first 28-day treatment cycle.

In certain embodiments, the paclitaxel and carboplatin are administered separately on a predetermined day as sequential single doses of each compound. For example, paclitaxel can be administered over a period of 2, 3, or 4 hours, followed by carboplatin over a period of 30 minutes, 60, minutes, or 120 minutes. Alternatively, carboplatin can be administered over a period of 30 minutes, 60, minutes, or 120 minutes, followed by paclitaxel over a period of 2, 3, or 4 hours.

In other embodiments, the paclitaxel and carboplatin are administered separately on a predetermined day as sequential multiple doses of each compound. For example, paclitaxel can be administered over a period of 1 hour, followed by carboplatin over a period of 30 minutes, followed by paclitaxel over a period of 1 hours, and followed again by paclitaxel over a period of 30 minutes. The order of administration of the paclitaxel and carboplatin can be reversed.

In still other embodiments, the paclitaxel and carboplatin can be administered together on a predetermined day as single dose of both compounds. For example, the paclitaxel and carboplatin can be combined and administered simultaneously over a period of 2, 3, 4, or 5 hours.

The dosage schedule may further comprise one or more of 28-day maintenance cycles. In one embodiment, the 28-day maintenance cycles comprises administering the Hsp90 inhibitor on alternating days for at least 21 days during each 28-day maintenance cycle. The Hsp90 inhibitor is administered in an amount of about 50 mg/m² to about 150 mg/m², or about 50 mg/m² to about 100 mg/m², or about 75 mg/m² to about 100 mg/m², or about 100 mg/m² during each 28-day maintenance cycle.

### Pharmaceutical Compositions

In some embodiments, the method comprises the administration of the Hsp90 inhibitor in a pharmaceutical composition having at least one pharmaceutically acceptable carrier, solvent, adjuvant or diluent.

The Hsp90 inhibitor described herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions described herein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

### EXAMPLES

The methods of the disclosure are illustrated further by the following examples, which are not to be construed as limiting the invention in scope or spirit to the specific procedures described in them.

### Example 1

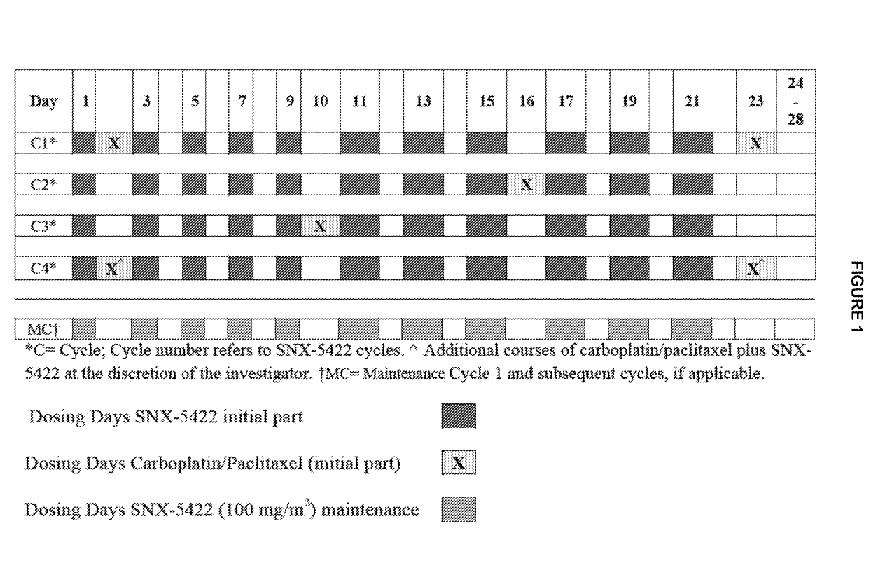

Starting on Day 1, SNX-5422 was dosed once every other day for 21 days (11 doses), followed by a 7-day drug free period. Patients received carboplatin and paclitaxel once every 21 days starting on Day 2 of SNX-5422 Cycle 1. Paclitaxel (175 mg/m²) was administered I.V. over 3 hours followed by administration of carboplatin (AUC 5) I.V. over 30-60 minutes. A total of 4 courses of paclitaxel and carboplatin were administered during 3 cycles of SNX-5422. Two additional optional courses may be administered (e.g., for a maximum of 6 courses) during the subsequent cycle of SNX-5422 (Cycle 4). Carboplatin and paclitaxel were not dosed on the same day as SNX-5422, and the performed dosing schedule is disclosed in FIG. 1.

### Example 2

### Treatment Cycles

Starting on Day 1, SNX-5422 was dosed once every other day for 21 days in patients with cancer. The performed dosing schedule is disclosed in FIG. 1. SNX-5422 dose was escalated as follows.

TABLE 1
SNX-5422 Dose Escalation Schedule

| Dose Level | SNX-5422 dose (qod) | |
|---|---|---|
| 1 | 50 | mg/m² P.O. |
| 2 | 75 | mg/m² P.O. |
| 3 | 100 | mg/m² P.O. |

Doses of SNX-5422 were increased until dose-limiting toxicity (DLT) is observed, and the maximum tolerated dose (MTD) of SNX-5422 given in combination with carboplatin and paclitaxel was identified. Dose escalation did not exceed a dose level of 100 mg/m² every other day (qod), which was the previously-established single agent MTD for SNX-5422. DLT is defined as adverse events (AEs) or laboratory abnormalities of Common Terminology Criteria for Adverse Events (CTCAE version 4.03).

Patients received carboplatin and paclitaxel once every 21 days for 4 courses and may receive a maximum of 6. The 4 courses of carboplatin and paclitaxel were administered during the first 3 cycles of SNX-5422, and the optional 2 courses during Cycle 4 as presented in FIG. 1. Paclitaxel and carboplatin were dosed as noted in Table 2:

TABLE 2
Paclitaxel and Carboplatin Dose Schedule

| Cycle Number | Dosing day | Dosing window |
|---|---|---|
| 1* | 2 | If necessary, chemo may be |
| | | administered on Day 4 |
| | 23 | ±1 Day |
| 2 | 16 | ±2 Day** |
| 3 | 10 | ±2 Day** |
| 4\hat{ } | 2 | If necessary, chemo may be |
| | | administered on Day 4 |
| | 23 | ±1 Day |

*SNX-5422 is dosed starting on Day 1 of each cycle.
**Preferably on Days 14 or 18 of Cycle 2, and Days 8 or 12 of Cycle 3 to avoid carboplatin and paclitaxel being dosed on the same day as SNX-5422.
\hat{ }Additional courses of carboplatin/paclitaxel plus SNX-5422

These dosing days of carboplatin and paclitaxel have been selected so that dosing of chemotherapy is initiated early and occurs after SNX-5422 dosing. If due to circumstances, dosing of chemotherapy coincides with SNX-5422 dosing, in one embodiment, the SNX-5422 dose may be held and resumed on the day post-chemotherapy.

Paclitaxel (175 mg/m²) will be administered I.V. over 3 hours followed by administration of carboplatin (AUC 5) I.V. over 30-60 minutes. The carboplatin dose is calculated using a modified Calvert formula as follows:

Total dose (mg)=(Target AUC)×(Creatinine Clearance+25)=5×(CLcreat+25).

wherein:

Carboplatin target AUC=5 mg/mL×min;

Creatinine clearance can either be measured or estimated using the Cockroft-Gault formula (Cockroft DW, Gault MH. Prediction of creatinine clearance from serum creatinine. Nephron 16(1): 31-41 (1976)) as follows:

CLcreat=(140−age)×body mass[×0.85 if female])/72×creatinine, where

age is given in years, body mass in kg, and creatinine in mg/dL.

The maximum dose of carboplatin (AUC=5) administered is not to exceed 750 mg.

### Maintenance Cycles

Patients treated with SNX-5422 plus paclitaxel and carboplatin with at, least stable disease received maintenance therapy with 100 mg/m² SNX-5422 qod, 21 out of 28 days. Depending on the type of, relationship and severity, patients who experienced toxicity during the maintenance part received the reduced dose of 75 or 87.5 mg/m² of SNX-5422.

### Example 3

Eligible patients that had advanced NSCLC (EGFR wild-type or non-sensitizing mutation, ALK wild-type) or extensive stage SCLC and up to one prior line of chemotherapy were administered SNX-5422, carboplatin, and paclitaxel according to Example 2. For example, patients received paclitaxel (175 mg/m²) and carboplatin (AUC 5) q3w up to 4 courses and SNX-5422 qod (starting at 50 mg/m²), 21 of 28 days, with a standard 3+3 dose escalation rule during the combination followed by SNX-5422 (100 mg/m² qod) monotherapy for maintenance until disease progression.

The SNX-5422 Maximum Tolerated Dose was determined at 100 mg/m² for the combination with one grade 3 DLT of diarrhea. Adverse events possibly related to the combination in a 2 pts were diarrhea, nausea, fatigue, neutropenia, alopecia, mostly graded 1 or 2, except for grade 3 neutropenia (2), diarrhea (2), and nausea (1). Of 18 NSCLC patients evaluable for objective response, 7 patients (39%) had partial response, 10 patients (56%) had stable disease. Of 3 SCLC patients, 2 patients (67%) had stable disease and 1 patient (33%) had partial response. In addition, 6 out of 9 (67%) patients receiving only 4 courses of SNX-5422, carboplatin, and paclitaxel (i.e., four 28-day treatment cycles) progressed onto maintenance cycle. In contrast, when patients with stage IIIB/IV NSCLC were treated with carboplatin and paclitaxel only, without SNX-5422, about 25% patients had partial response rate and 24% patients had stable disease as best response; and only 57% of patients completed 4 courses of chemo (Socinski M A et al. J Clin Oncol. 20(5):1335-43 (2002)).

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method for treating cancer in a subject in need thereof, the method comprising administering to the subject:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(am inocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, wherein the Hsp90 inhibitor is administered in an amount of about 50 mg/m² to about 150 mg/m²;
b) carboplatin administered in an amount sufficient to result in a target Area under Curve (AUC) of about 2 to about 7; and
c) paclitaxel administered in an amount of about 100 mg/m² to about 225 mg/m².

2. A method for treating cancer in a subject in need thereof, the method comprising administering a therapeutically effective amount of:
a) an Hsp90 inhibitor, which is 4-(6,6-Dimethyl-4-oxo-3-trifluoromethyl-4,5,6,7-tetrahydro-indazol-1-yl)-2-(trans-4-hydroxy-cyclohexylamino)-benzamide, trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof;
b) carboplatin; and
c) paclitaxel,
to the subject on a dosage schedule, wherein the dosage schedule comprises four or six 28-day treatment cycles, and wherein:
(i) the Hsp90 inhibitor is administered on alternating days, for at least 21 days during each 28-day treatment cycle; and
(ii) carboplatin and paclitaxel are administered every 21±2 days starting on day 2 or day 4 of the first 28-day treatment cycle.

3. A method of claim 2, wherein the Hsp90 inhibitor is administered in an amount of about 50 mg/m² to about 150 mg/m²; carboplatin is administered in an amount sufficient to result in a target Area under Curve (AUC) of about 2 to about 7; and paclitaxel is administered in an amount of about 100 mg/m² to about 225 mg/m².

4. A method of claim 1, wherein the Hsp90 inhibitor is trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate.

5. A method of claim 1, wherein the Hsp90 inhibitor is administered in an amount of about 50 mg/m² to about 100 mg/m².

6. A method of claim 1, wherein the Hsp90 inhibitor is administered in an amount that gradually increases from about 50 mg/m² to about 100 mg/m².

7. A method of claim 1, wherein the Hsp90 inhibitor is administered in an amount of about 100 mg/m².

8. A method of claim 1, wherein carboplatin is administered in an amount sufficient to result in a target Area under Curve (AUC) of about 3 to about 6.

9. A method of claim 1, wherein paclitaxel is administered in an amount of about 100 mg/m² to about 200 mg/m².

10. A method of claim 1, wherein the Hsp90 inhibitor is administered in an amount of about 100 mg/m²; carboplatin is administered in an amount sufficient to result in target Area under Curve (AUC) of about 5; and paclitaxel is administered in an amount of about 170 mg/m² to about 180 mg/m².

11. A method of claim 2, wherein the dosage schedule comprises four 28-day treatment cycles.

12. A method of claim 2, wherein the dosage schedule further comprises one or more of 28-day maintenance cycles.

13. A method of claim 12, wherein the 28-day maintenance cycles comprises administering the Hsp90 inhibitor on alternating days for at least 21 days during each 28-day maintenance cycle.

14. A method of claim 12, wherein the Hsp90 inhibitor is administered in an amount of about 100 mg/m² during each 28-day maintenance cycle.

15. A method of claim 2, wherein carboplatin and paclitaxel are administered starting on day 2 of the first 28-day treatment cycle.

16. A method of claim 1, wherein cancer is lung, esophageal, ovarian, head-and-neck, mesothelioma, melanoma, testicular, stomach, bladder, uterine, colon, prostate, renal cell, pancreatic, or neuroendocrine cancer.

17. A method of claim 1, wherein cancer is lung cancer.

18. A method of claim 17, wherein lung cancer is non-small cell lung cancer (NSCLC).

19. A method of claim 2, wherein cancer is lung cancer.

20. A method of claim 19, wherein lung cancer is non-small cell lung cancer (NSCLC).
